(19) United States
(12) Patent Application Publication
(10) Pub. No.: US 2011/0301382 A1
(43) Pub. Date: Dec. 8, 2011

(54) Processes for Preparing 1,3-Dinitro-5-(Pentafluorosulfanyl)Benzene and its Intermediates

(75) Inventors: Teruo Umemoto, Westminster, CO (US); Junichi Chika, Broomfield, CO (US)

(73) Assignee: UBE INDUSTRIES, LTD., Tokyo (JP)

(21) Appl. No.: 12/961,750

(22) Filed: Dec. 7, 2010

Related U.S. Application Data
(60) Provisional application No. 61/267,304, filed on Dec. 7, 2009.

(51) Int. Cl. C07C 63/10; C07C 381/00

(52) U.S. Cl. 562/432; 568/74

(57) ABSTRACT

New processes for preparing 1,3-dinitro-5-(pentafluorosulfanyl)benzene starting from 4-(pentafluorosulfanyl)toluene or (pentafluorosulfanyl)benzene are disclosed. The useful intermediates are also disclosed.

### TECHNICAL FIELD

The invention relates to processes for preparing 1,3-dinitro-5-(pentafluorosulfanyl)benzene and its intermediates.

### BACKGROUND OF THE INVENTION

1,3-Dinitro-5-(pentafluorosulfanyl)benzene is an important intermediate for production of 1,3-diamino-5-(pentafluorosulfanyl)benzene, a monomer in the production of useful polyimide polymers containing an SF₅ moiety (see U.S. Pat. No. 5,220,070). These SF₅-containing polymers exhibit high glass transition temperature, high density, low solubility, and low dielectric properties, and are used to prepare semi-permeable membranes, wire coatings, and films. These polymers are also useful in various electronic, aerospace, and piezoelectric applications (see U.S. Pat. No. 5,302,692).

Conventional production methods for 1,3-dinitro-5-(pentafluorosulfanyl)benzene include a reaction of bis(3,5-dinitrophenyl)disulfide with silver difluoride (see U.S. Pat. No. 5,220,070). However, this method results in a very low yield (7%) and requires use of an expensive starting material [bis(3,5-dinitrophenyl)disulfide] and a very expensive fluorinating reagent [silver difluoride (AgF₂)]. In general, reagents to perform silver-based reactions are cost prohibitive especially when performed at an industrial scale. Therefore, these problems make it impractical to prepare 1,3-dinitro-5-(pentafluorosulfanyl)benzene via conventional methods, especially at an industrial scale.

Pentafluorosulfanyl group (SF₅) is a strong electron-withdrawing group. SF₅ on a benzene ring is hydrolyzed by severe acidic conditions, as it has been reported that (pentafluorosulfanyl)benzene is hydrolyzed to benzenesulfonyl fluoride by heating at 100° C. in 100% sulfuric acid [see J. Am. Chem. Soc., Vol. 84 (1962), pp. 3064-3072]. Thus, there are no reports of nitration of 3-(pentafluorosulfanyl)nitrobenzene to give 1,3-dinitro-5-(pentafluorosulfanyl)benzene in the literature, as the benzene ring is greatly deactivated by both the strong electron-withdrawing effects of SF₅ and NO₂ groups, and the severe acidic conditions necessary for the nitration event. Generally, these conditions result in decomposition of the SF₅ group [for mono-nitration of (pentafluorosulfanyl)benzene; see, for example, J. Am. Chem. Soc., Vol. 84 (1962), pp. 3064-3072 and Organic Letters, Vol. 6 (2004), pp. 2417-2419].

The present invention is directed toward overcoming the problems discussed above.

### SUMMARY OF THE INVENTION

The present invention provides a unified process, as well as the individual steps in the process, for the preparation of 1,3-dinitro-5-(pentafluorosulfanyl)benzene from 4-(pentafluorosulfanyl)toluene (as a starting material). Embodiments of the invention include: reacting 4-(pentafluorosulfanyl)toluene with a nitrating agent to form 2-nitro-4-(pentafluorosulfanyl)toluene and then to form 2,6-dinitro-4-(pentafluorosulfanyl)toluene, oxidizing the resulting 2,6-dinitro-4-(pentafluorosulfanyl)toluene to form 2,6-dinitro-4-(pentafluorosulfanyl)benzoic acid, and decarboxylating the resulting 2,6-dinitro-4-(pentafluorosulfanyl)benzoic acid to form 1,3-dinitro-5-(pentafluorosulfanyl)benzene.

The present invention also provides new useful intermediate compounds as presented by formula (A):

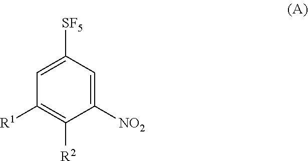

in which R¹ is a hydrogen atom or a nitro group and R² is a methyl group or a carboxyl group.

The present invention provides another new useful intermediate which is a 1:1 compound of 2,6-dinitro-4-(pentafluorosulfanyl)benzoic acid and isopropanol.

This invention also provides processes for preparing 1,3-dinitro-5-(pentafluorosulfanyl)benzene by reacting (pentafluorosulfanyl)benzene with a nitrating agent to form 3-(pentafluorosulfanyl)nitrobenzene and then to form 1,3-dinitro-5-(pentafluorosulfanyl)benzene. This invention also includes a process of reacting 3-(pentafluorosulfanyl)nitrobenzene with a nitrating agent to form 1,3-dinitro-5-(pentafluorosulfanyl)benzene.

These and various other features and advantages of the invention will be apparent from a reading of the following detailed description and a review of the appended claims.

### DETAILED DESCRIPTION OF THE INVENTION

Embodiments of the present invention provide industrially useful processes for producing 1,3-dinitro-5-(pentafluorosulfanyl)benzene [compound (I) in Table 1]. The (pentafluorosulfanyl)benzene is a useful intermediate for preparing 1,3-diamino-5-(pentafluorosulfanyl)benzene (see U.S. Pat. No. 5,220,070, incorporated herein by reference; and also see Reference Example 2, Examples Section), which is a useful monomer for production of SF_{S}-containing polyimide polymers (see U.S. Pat. No. 5,302,692, incorporated herein by reference). Unlike conventional methods in the art, the processes of the present invention utilize relatively inexpensive reagents and conveniently prepared 4-(pentafluorosulfanyl)toluene, (pentafluorosulfanyl)benzene, or 3-(pentafluorosulfanyl)nitrobenzene as a starting material. These starting materials are also inexpensive since they are prepared using relatively inexpensive materials and reagents, for example, by reaction of di(p-tolyl)disulfide or diphenyl disulfide with chlorine (Cl₂) and potassium fluoride, followed by reaction with a fluoride source such as zinc difluoride in the presence or absence of a halogen such as Cl₂ (see U.S. Pat. No. 7,592,491 B2, incorporated herein by reference). 3-(Pentafluorosulfanyl)nitrobenzene is conveniently prepared by nitration of (pentafluorosulfanyl)benzene in high yield (see J. Am. Chem. Soc., Vol. 84 (1962), pp. 3064-3072; Organic Letters, Vol. 6 (2004), pp. 2417-2419; and also see Reference Example 1). Therefore, according to the processes of the present invention, 1,3-dinitro-5-(pentafluorosulfanyl)benzene is produced at low cost in comparison to the prior art methodology.

Embodiments of the invention include processes which comprise (see Scheme I) (step 1) reacting 4-(pentafluorosulfanyl)toluene (II) with a nitrating agent to form 2-nitro-4-(pentafluorosulfanyl)toluene (III), (step 2) reacting the resulting 2-nitro-4-(pentafluorosulfanyl)toluene (III) with a nitrating agent to form 2,6-dinitro-4-(pentafluorosulfanyl)toluene (IV), (step 3) oxidizing the resulting 2,6-dinitro-4-(pentafluorosulfanyl)toluene (IV) to form 2,6-dinitro-4-(pentafluorosulfanyl)benzoic acid (V), and (step 4) decarboxylating the resulting 2,6-dinitro-4-(pentafluorosulfanyl)benzoic acid (V) to form 1,3-dinitro-5-(pentafluorosulfanyl)benzene (I).

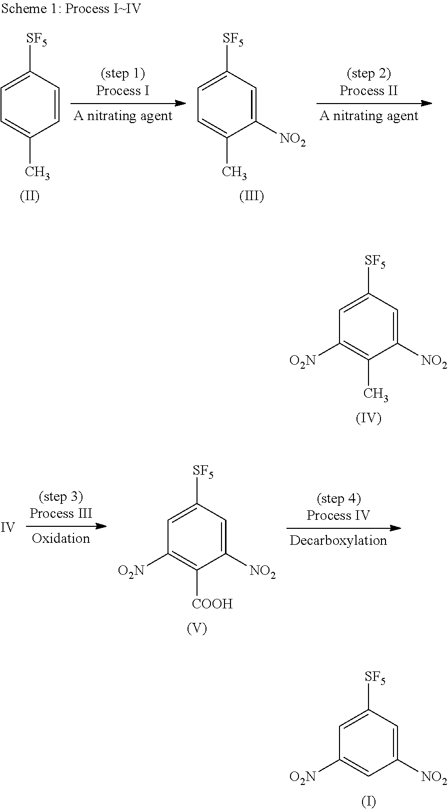

Table 1 provides chemical names and corresponding structures as well as their formula number for reference herein.

TABLE 1
Formulas I~VII

| | | Compound |
|---|---|---|
| Chemical Name | Structure | number |
| 1,3-Dinitro-5- (pentafluorosulfanyl)benzene | 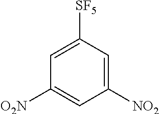 | I |
| 4-(Pentafluorosulfanyl)toluene | 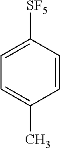 | II |
| 2-Nitro-4- (pentafluorosulfanyl)toluene | 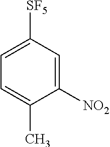 | III |
| 2,6-Dinitro-4- (pentafluorosulfanyl)toluene | 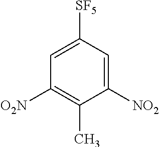 | IV |
| 2,6-Dinitro-4- (pentafluorosulfanyl)benzoic acid | 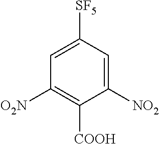 | V |
| (Pentafluorosulfanyl)benzene | 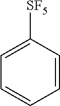 | VI |
| 3- (Pentafluorosulfanyl)nitro- benzene | 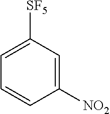 | VII |

### Process I (Scheme 1)

Process I includes reacting compound (II) with a nitrating agent to form compound (III). Known nitrating agents can be used, such as nitric acid, a mixture of nitric acid and an acid, fuming nitric acid, a mixture of fuming nitric acid and an acid, nitronium tetrafluoroborate, nitronium trifluoromethanesulfonate, and so on. Nitronium trifluoromethanesulfonate can be in situ prepared by reaction of nitric acid or fuming nitric acid with trifluoromethanesulfonic anhydride [see, for example, J. Am. Chem. Soc., Vol. 115 (1993), p.p. 2156-2164, incorporated herein by reference]. Among nitrating agents, nitric acid, a mixture of nitric acid and an acid, fuming nitric acid, and a mixture of fuming nitric acid and an acid are exemplified preferably from a viewpoint of cost and product yield.

Acids for use as part of a nitrating agent, such as a mixture of nitric acid and an acid and a mixture of fuming nitric acid and an acid, are exemplified by strong acids, such as sulfuric acid, fuming sulfuric acid, chlorosulfonic acid, fluorosulfonic acid, trifluoromethanesulfonic acid, tetrafluoroboric acid, hexafluorophosphoric acid, and so on. Among these acids, sulfuric acid and fuming sulfuric acid are exemplified preferably from a viewpoint of cost and product yield.

Reaction conditions of Process I are optimized to obtain economically good yields of product. The amount of nitrating agent can be preferably selected in the range of about 1 mol to about 5 mol, more preferably about 1 mol to about 3 mol, against 1 mol of compound (II) to obtain a good yield of compound (III). These values are calculated based on the fact that 1 mol of a nitrating agent is the amount of nitrating agent that generates 1 mol of NO₂⁺ species.

The Process I reaction can be conducted in the absence or presence of solvent. When a solvent is required, suitable solvents include acids, hydrocarbons, halocarbons, nitro compounds, and so on. Illustrative acids are sulfuric acid, fuming sulfuric acid, trifluoromethanesulfonic acid, fluorosulfonic acid, chlorosulfonic acid, trifluoroacetic acid, acetic acid, phosphoric acid, and so on. As mentioned above, strong acids also act as a part of the nitrating agents. Illustrative hydrocarbons include straight, branched and cyclic hexane, heptane, octane, nonane, decane, undecane, dodecane, and so on. Illustrative halocarbons include dichloromethane, chloroform, tetrachlorocarbon, dichloroethane, trichloroethane, tetrachloroethane, and so on. Illustrative nitro compounds are nitromethane and so on.

In order to obtain relatively good yields of product in Process I, the reaction temperature can be selected in the range of about −30° C. to about +80° C. More preferably, the reaction temperature can be selected in the range of about −20° C. to about +60° C., furthermore preferably, about −10° C. to about +50° C.

Product compound (III) may be isolated by normal post-treatment procedures including extraction, precipitation, distillation, or crystallization, however, compound (III) may be used for the next reaction (Process II) without isolation or without purification.

### Process II (Scheme I)

Process II includes reacting compound (III) with a nitrating agent to form compound (IV). Process II is similar to Process I except for the amount of a nitrating agent used and the reaction temperature. In order to obtain a good yield of compound (IV), the amount of nitrating agent can be preferably selected in the range of about 1 mol to about 10 mol against 1 mol of compound (III). The reaction temperature is preferably selected in the range of about 0° C. to about +100° C. More preferably, the reaction temperature can be selected in the range of about 0° C. to about +80° C.

### Process III (Scheme I)

Process III includes oxidizing compound (IV) with an oxidizing agent to form compound (V). Oxidizing agents herein can include: chromium(VI) oxide (CrO₃); salts of dichromates or their hydrates, such as sodium dichromate (Na₂Cr₂O₇), sodium dichromate dihydrate (Na₂Cr₂O₇.2H₂O), potassium dichromate (K₂Cr₂O₇) and so on; salts of permanganates or their hydrates such as sodium permanganate (NaMnO₄), sodium permanganate monohydrate (NaMnO₄.H₂O), potassium permanganate (KMnO₄), and so on; salts of persulfates or their hydrates such as sodium persulfate (NaS₂O₈), potassium persulfate (KS₂O₈), and so on; salts of periodates or their hydrates such as sodium periodate (NaIO₄), potassium periodate (KIO₄), and so on. Among them, there are preferably exemplified chromium(VI) oxide (CrO₃); salts of dichromates or their hydrates such as sodium dichromate (Na₂Cr₂O₇), sodium dichromate dihydrate (Na₂Cr₂O₇.2H₂O), potassium dichromate (K₂Cr₂O₇) and so on; salts of permanganates or their hydrates such as sodium permanganate (NaMnO₄), sodium permanganate monohydrate (NaMnO₄.H₂O), potassium permanganate (KMnO₄), and so on. Chromium(VI) oxide (CrO₃) and salts of dichromates or their hydrates such as sodium dichromate (Na₂Cr₂O₇), sodium dichromate dihydrate (Na₂Cr₂O₇.2H₂O), and potassium dichromate (K₂Cr₂O₇) are exemplified furthermore preferably.

The oxidation can be carried out in a solvent such as water, sulfuric acid, fuming sulfuric acid, nitric acid, fuming nitric acid, acetic acid, acetic anhydride, trifluoroacetic acid, trifluoroacetic anhydride, methanesulfonic acid, methanesulfonic anhydride, trifluoromethanesulfonic acid, trifluoromethanesulfonic anhydride, phosphoric acid, and so on, and mixtures thereof. Among these solvents, sulfuric acid, fuming sulfuric acid, trifluoroacetic acid, and mixtures thereof are further preferably exemplified.

With regard to solvent mixtures described herein, any two or more enumerated solvents can be combined to substitute for any one solvent. This also relates to other noted “mixtures”, the mixture can be two or more of the listed items to substitute for any one of the same listed items. When volume or amount is an issue, the mixture of ingredients substitutes the same volume or amount as the total for one ingredient.

Reaction conditions of Process III are optimized to obtain economically good yields of product. The amount of an oxidizing agent can be preferably selected in the range of about 2 mol to about 20 mol against 1 mol of compound (IV) to obtain a good yield of compound (V). The reaction temperature is preferably selected in the range of about 0° C. to about +100° C. More preferably, the reaction temperature can be selected in the range of about 0° C. to about +80° C.

Product compound (V) may be isolated by normal post-treatment such as extraction and/or crystallization, or in some embodiments compounds (V) may be used for the next reaction (Process IV) without isolation or without purification.

Compound (V) forms a stable 1:1 crystalline compound with isopropanol (see Example 6). This makes the isolation and purification of the compound (V) easy.

### Process IV (Scheme I)

Process IV includes decarboxylating compound (V) to form compound (I). The decarboxylation can be carried out by heating compound (V) under conditions such as neutral, basic, or acidic conditions. The decarboxylation of compound (V) can be carried out in the presence or absence of solvent. In order to make the reaction predictable, solvent is typically used. Exemplified solvents include water, alcohols, ethers, alkanes, haloalkanes, aromatics, nitriles, esters, ketones, amides, acids, and so on, and mixtures thereof. Exemplified alcohols include methanol, ethanol, propanol, isopropanol, butanol, isobutanol, sec-butanol, tert-butanol, ethylene glycol, propylene glycol, and so on. Exemplified ethers include diethyl ether, dipropyl ether, diisopropyl ether, tetrahydrofuran, dimethoxyethane, dioxane, and so on. Exemplified alkanes include straight, branched, and cyclic hexane, heptane, octane, nonane, decane, and so on. Exemplified haloalkanes include dichloromethane, chloroform, carbon tetrachloride, dichloroethane, and so on. Exemplified aromatics include benzene, toluene, xylene, chlorobenzene, and so on. Exemplified nitriles include acetonitrile, propionitrile, and so on. Exemplified esters include methyl acetate, ethyl acetate, methyl propionate, and so on. Exemplified ketones include acetone, methyl ethyl ketone, diethyl ketone, and so on. Exemplified amides include formamide, methyl formamide, dimethyl formamide, dimethyl acetamide, and so on. Exemplified acids include formic acid, acetic acid, propionic acid, phosphoric acid, sulfuric acid, and so on. Among these solvents, water, alcohols, ethers, alkanes, haloalkanes, aromatics, nitriles, and mixtures thereof are preferably exemplified, and water, alcohols, ethers, and mixtures thereof are more preferably exemplified due to cost and product yield considerations.

Decarboxylation of 2,4-dinitro-4-(pentafluorosulfanyl)benzoic acid (V) can be carried out under conditions such as under neutral, basic, or acidic conditions, for example, neutral, basic and acidic conditions each relate to a benzoic acid form and its conjugated base form. In one embodiment, conditions including a conjugated base of the benzoic acid (V) are preferable because the decarboxylation takes place at a lower temperature than at the other conditions. The conjugated base of the benzoic acid (V) is 2,6-dinitro-4-(pentafluorosulfanyl)benzoate anion [ArCOO⁻ wherein Ar=2,6-dinitro-4-(pentafluorosulfanyl)phenyl group]. The decarboxylation under conditions including the conjugated base can be conducted by mixing the benzoic acid (V) with a base(s). As a base, normally well-known bases can be exemplified, for example, an alkali metal hydroxide such as LiOH, NaOH, KOH, CsOH, and so on; an alkali earth metal hydroxide or oxide such as Mg(OH)₂, MgO, Ca(OH)₂, CaO, and so on; a carbonate such as Li₂CO₃, LiHCO₃, Na₂CO₃, NaHCO₃, K₂CO₃, KHCO₃, and so on; ammonia (NH₃); an amine such as methylamine, dimethylamine, trimethylamine, ethylamine, diethylamine, triethylamine, propylamine, butylamine, and so on; a pyridine such as pyridine, methylpyridine, dimethylpyridine, trimethylpyridine, and so on; a tetraalkylammonium hydroxide such as tetramethylammonium hydroxide, tetraethylammonium hydroxide, tetrapropylammnoium hydroxide, tetrabutylammonium hydroxide, benzyltrimethylammonium hydroxide, and so on; and a carboxylate salt such as sodium acetate, potassium acetate, ammonium acetate, and so on. Among these bases, alkali metal hydroxides can be used preferably due to cost and product yield consideration. The amount of base used can be selected in the range of a catalytic amount to a large excess against the amount of the benzoic acid (V). As exemplified in Reaction Scheme 1, the benzoic acid (V) can first be converted by a base to 2,6-dinitro-4-(pentafluorosulfanyl)benzoate salt (V′), which undertakes decarboxylation to give product (I). Reaction Scheme 1 shows a case wherein a base is NaOH and a solvent is water. As shown in Reaction Scheme 1, NaOH can be regenerated by the decarboxylation reaction.

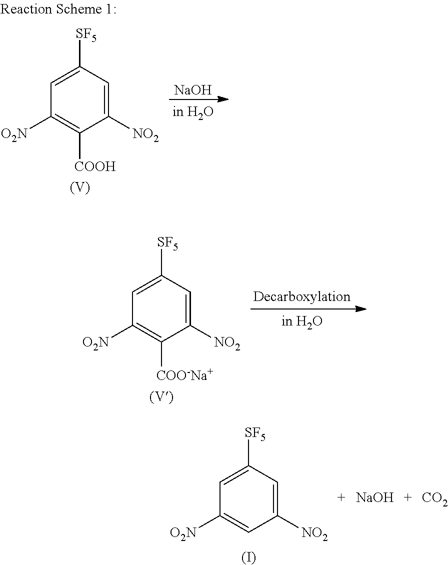

Reaction conditions of Process IV are optimized to obtain economically good yields of product. The reaction temperature for the decarboxylation is preferably selected in the range of about 0° C. to about +200° C. More preferably, the temperature can be selected in the range of about 0° C. to about +150° C., furthermore preferably, about 0° C. to about +130° C. Since the decarboxylation temperature of the benzoic acid (V) depends on the reaction conditions, a suitable temperature may be selected for each condition. The decarboxylation reaction under conditions including a conjugated base of the benzoic acid (V) may take place at relatively low temperature.

Embodiments of the present invention also include steps 1 and 2 conducted as a “one-pot” reaction (Scheme II, Process V), i.e. all materials for steps 1 and 2 are put together in a non-sequential manner.

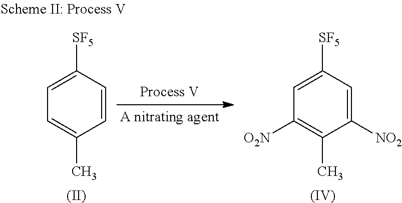

### Process V (Scheme II)

Process V includes reacting compound (II) with a nitrating agent to form compound (IV). Process V is similar to Process I except for the amount of nitrating agent used and the reaction temperature. In order to get an economically good yield of product compound (IV), the amount of a nitrating agent is preferably selected in the range of about 2 mol to about 10 mol against 1 mol of compound (II). The reaction temperature is preferably selected in the range of about −30° C. to about +100° C. and, more preferably, about −10° C. to about +80° C.

The present invention provides new useful intermediate compounds as presented by formula (A):

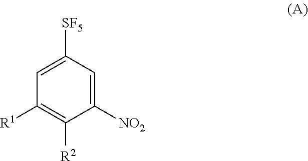

in which R¹ is a hydrogen atom or a nitro group and R² is a methyl group or a carboxyl group. The (pentafluorosulfanyl)benzene derivative of formula (A) is preferably selected from a group consisting of 2,6-dinitro-4-(pentafluorosulfanyl)benzoic acid (V), 2,6-dinitro-4-(pentafluorosulfanyl)toluene (IV), and 2-nitro-4-(pentafluorosulfanyl)toluene (III).

The present invention also includes a 1:1 compound of 2,6-dinitro-4-(pentafluorosulfanyl)benzoic acid (V) and isopropanol as a new useful intermediate compound.

The present invention includes processes which comprise reacting 3-(pentafluorosulfanyl)nitrobenzene (VII) with a nitrating agent to form 3,5-dinitro-(pentafluorosulfanyl)benzene (I) (Scheme III, Process VI).

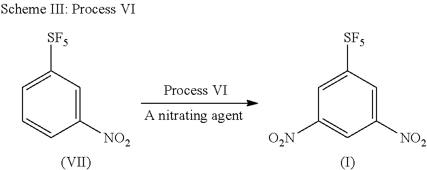

### Process VI (Scheme III)

Process VI includes reacting compound (VII) with a nitrating agent to form compound (I). Compound (VII) as a starting material is prepared by a method reported in the literature (see J. Am. Chem. Soc., Vol. 84 (1962), pp. 3064-3072; Organic Letters, Vol. 6 (2004), pp. 2417-2419, incorporated herein by reference; and also see Reference Example 1).

Known nitrating agents can be used, such as nitric acid, a mixture of nitric acid and an acid, fuming nitric acid, a mixture of fuming nitric acid and an acid, nitronium tetrafluoroborate, nitronium trifluoromethanesulfonate, and so on. Nitronium trifluoromethanesulfonate can be in situ prepared by reaction of nitric acid or fuming nitric acid with trifluoromethanesulfonic anhydride (see, for example, J. Am. Chem. Soc., Vol. 115 (1993), p.p. 2156-2164, incorporated herein by reference). Among nitrating agents, nitric acid, a mixture of nitric acid and an acid, fuming nitric acid, and a mixture of fuming nitric acid and an acid are exemplified preferably from a viewpoint of relative expense and product yield.

As an acid usable as a part of a nitrating agent, such as a mixture of nitric acid and an acid and a mixture of fuming nitric acid and an acid, there are exemplified strong acids such as sulfuric acid, fuming sulfuric acid, chlorosulfonic acid, fluorosulfonic acid, trifluoromethanesulfonic acid, tetrafluoroboric acid, hexafluorophosphoric acid, and so on. Among these acids, sulfuric acid and fuming sulfuric acid are exemplified preferably from a viewpoint of relative expense and product yield.

Reaction conditions of Process VI are optimized to obtain economically good yields of product. The amount of a nitrating agent can be selected in the range of about 1 mol to about 20 mol against 1 mol of compound (VII) to obtain a good yield of compound (I).

The reaction of Process VI can be conducted in the absence or presence of solvent. When a solvent is required, suitable solvents include: acids, hydrocarbons, halocarbons, nitro compounds, and so on. Illustrative acids are sulfuric acid, fuming sulfuric acid, trifluoromethanesulfonic acid, fluorosulfonic acid, chlorosulfonic acid, and so on. As mentioned above, strong acids also act as a part of nitrating agents. Illustrative hydrocarbons are straight, branched, and cyclic hexane, heptane, octane, nonane, decane, undecane, dodecane, and so on. Illustrative halocarbons are dichloromethane, chloroform, tetrachlorocarbon, dichloroethane, trichloroethane, tetrachloroethane, and so on. Illustrative nitro compounds are nitromethane and so on. Among the solvents, acids are preferable, and sulfuric acid and fuming sulfuric acid are exemplified more preferably.

In order to obtain good yields of product in Process VI, the reaction temperature can be selected in the range of about 0° C. to about +120° C. More preferably, the reaction temperature can be selected in the range of about 0° C. to about +110° C., furthermore preferably, about 0° C. to about +100° C.

Embodiments of the present invention also include processes which comprise reacting (pentafluorosulfanyl)benzene (VI) with a nitrating agent to form 1,3-dinitro-5-(pentafluorosulfanyl)benzene (I) (Scheme IV, Process VII).

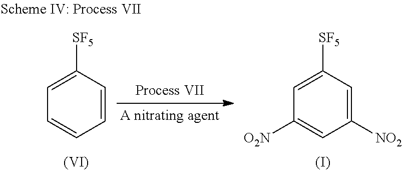

### Process VII (Scheme IV)

Process VII includes reacting compound (VI) with a nitrating agent to form compound (I). Compound (VI) is inexpensive since it is prepared using affordable materials and reagents, for example, by reaction of diphenyl disulfide with chlorine (Cl₂) and potassium fluoride, followed by reaction with a fluoride source such as zinc difluoride in the presence or absence of a halogen such as Cl₂ (see U.S. Pat. No. 7,592,491 B2, incorporated herein by reference).

Process VII is similar to Process VI except for compound (VI) used in place of compound (VII), the amount of a nitrating agent, and the reaction temperature. The amount of a nitrating agent can be preferably selected in the range of about 2 mol to about 20 mol against 1 mol of compound (VI) to obtain an economically good yield of compound (I). The reaction temperature is preferably selected in the range of about −10° C. to about +120° C. More preferably, the reaction temperature can be selected in the range of about −10° C. to about +110° C., furthermore preferably, about −10° C. to about +100° C.

According to the processes of the invention, 1,3-dinitro-5-(pentafluorosulfanyl)benzene (I) is produced at low cost in comparison to prior art methodology.

### EXAMPLES

The following examples are provided for illustrative purposes only and are not intended to limit the scope of the invention.

### Example 1

### Preparation of 2,6-dinitro-4-(pentafluorosulfanyl)toluene (IV): A one pot reaction of 4-(pentafluorosulfanyl)toluene (II)

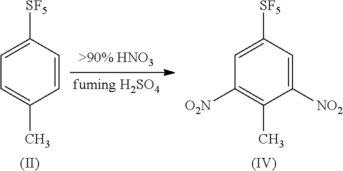

56.4 g of concentrated sulfuric acid, 56.4 g of fuming sulfuric acid (28% SO₃—H₂SO₄), and 17.5 g (278˜250 mmol) of >90% nitric acid are mixed in a 250 mL flask under cooling conditions with an ice bath. After removing the ice bath, 10.9 g (50.0 mmol) of 4-(pentafluorosulfanyl)toluene (II) was added dropwise into the mixture over an hour. Mild exothermic reaction occurred with addition of 4-(pentafluorosulfanyl)toluene (II). The temperature of the reaction mixture gradually increased and reached 35° C. when the addition was complete. After the addition, the reaction mixture was heated at 48-49° C. for 6 hours (h) and poured into ice (292 g). The resulting precipitates were collected by filtration and washed with water to give pale yellow solid, which was recrystallized from isopropanol to give 13.6 g (88% yield) of 2,6-dinitro-4-(pentafluorosulfanyl)toluene (IV) as light yellow crystals. Its physical properties and spectral data are shown as follows: M.p.; 107.8-108.9° C.: ¹H-NMR (300 MHz, CD₃CN, ppm) 8.51 (2H, s), 2.55 (3H, s): ¹³C-NMR (75 MHz, CDCl₃, ppm) 151.32 (quintet, J=23.1 Hz), 151.26, 131.54, 125.36 (t, J=4.7 Hz), 15.31: ¹⁹F-NMR (282 MHz, CD₃CN, ppm) 79.10 (1F, quintet, J=150 Hz), 62.62 (4F, d, J=150 Hz): IR (KBr, cm⁻¹) 3107, 2878, 2359, 1828, 1622, 1538, 1439, 1349, 1296, 1170, 1107, 908, 843, 795. Elemental analysis: Calcd for C₇H₅F₅N₂O₄S: C, 27.28%; H, 1.64%; N, 9.09%. Found: C, 27.31%; H, 1.64%; N, 8.70%.

### Example 2

### Preparation of 2-nitro-4-(pentafluorosulfanyl)toluene (III)

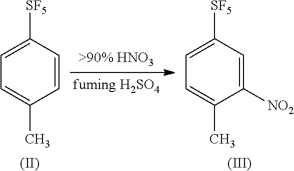

A mixture of 10 mL (238˜214 mmol) of >90% nitric acid and 10 mL of concentrated sulfuric acid was added dropwise into a stirred mixture of 21.8 g (100 mmol) of 4-(pentafluorosulfanyl)toluene (II) and 15.5 mL of concentrated sulfuric acid cooled on an ice bath. An exothermic reaction occurred. The addition took approximately 12 minutes (min). After addition, the reaction mixture was stirred for 3 h at 20° C., poured into ice-water (150 g), and extracted with dichloromethane. The organic layer was washed with water and then aqueous saturated sodium bicarbonate solution, and then dried with magnesium sulfate, and filtered. Evaporation of solvent gave an oily product, which was distilled under reduced pressure to give 25.2 g (96% yield) of 2-nitro-4-(pentafluorosulfanyl)toluene (III) as a light yellow oil. The physical properties and spectral data are shown in the following: M.p.; 29-30° C.: B.p.; 108° C./4.6 mmHg: ¹H-NMR (300 MHz, CD₃CN, ppm) 8.33 (1H, d, J=2.1 Hz), 7.95 (1H, dd, J=2.4 Hz, 8.6 Hz), 7.58 (1H, d, J=8.6 Hz), 2.57 (3H, s): ¹³C-NMR (75 MHz, CDCl₃, ppm) 151.64 (quintet, J=20.1 Hz), 137.84, 133.42, 129.95 (t, J=4.4 Hz), 122.78 (t, J=4.7 Hz), 20.22: ¹⁹F-NMR (282 MHz, CD₃CN, ppm) 81.84 (1F, quintet, J=150 Hz), 62.42 (4F, d, J=150 Hz): IR (neat, cm⁻¹) 3119, 2993, 2940, 2875, 1936, 1799, 1535, 1354, 847, 791. Elemental analysis: Calcd for C₇H₆F₅NO₂S: C, 31.95%; H, 2.30%; N, 5.32%. Found: C, 31.91%; H, 2.35%; N, 5.30%.

### Example 3

### Preparation of 2,6-dinitro-4-(pentafluorosulfanyl)toluene (IV)

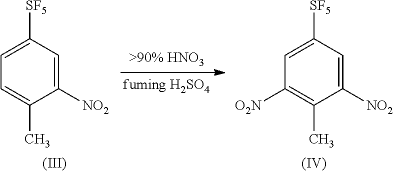

29.2 g of concentrated sulfuric acid, 29.2 g of fuming sulfuric acid (28% SO₃—H₂SO₄), and 11.2 g (178˜160 mmol) of >90% nitric acid were placed in a 100 mL flask. 2-Nitro-4-(pentafluorosulfanyl)toluene (III) (10.5 g, 40 mmol) was added into the 100 mL flask on an oil bath of 50° C. The reaction mixture was stirred on an oil bath of 50° C. for 7 h, and poured into ice-water (234 g) and extracted with dichloromethane. The organic layer was washed with water and then aqueous saturated sodium bicarbonate solution, dried with magnesium sulfate, and filtered. Evaporation of solvent from the filtrate gave 10.7 g of solid, which was recrystallized from isopropanol to give 9.82 g (80% yield) of 2,6-dinitro-4-(pentafluorosulfanyl)toluene (IV) as light yellow crystals. The physical properties and spectral data are shown in Example 1.

### Example 4

### Preparation of 2,6-dinitro-4-(pentafluorosulfanyl)benzoic acid (V) using sodium dichromate dihydrate as an oxidizing agent

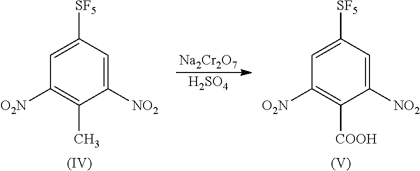

Sodium dichromate dihydrate (Na₂Cr₂O₇.2H₂O) (6.71 g, 22.5 mmol) was added portion by portion over 3.5 h to a stirred mixture of 3.08 g (10 mmol) of 2,6-dinitro-4-(pentafluorosulfanyl)toluene (IV) and 30 mL of concentrated sulfuric acid on an oil bath of 40° C. After the addition, the reaction mixture was stirred on an oil bath of 40° C. for 2.5 h and poured into ice-water (220 g), followed by extraction with ethyl acetate. The organic layer was washed with aqueous sodium chloride solution, dried with magnesium sulfate, and filtered. Evaporation of solvent from the filtrate gave a solid (2.56 g; crude yield 76%). The solid was dissolved in diethyl ether and toluene was added into the ether solution. After the ether was removed from the mixture under the reduced pressure, a small amount of hexane was added to the mixture. The mixture was stirred on an ice bath for 30 min and the resulting precipitates were collected by filtration, washed with a cold 1:1 mixture of toluene and hexane, and dried in vacuum, giving 2.03 g (yield 60%) of 2,6-dinitro-4-(pentafluorosulfanyl)benzoic acid (V) as white crystals. Its physical properties and spectral data are shown in the following: M.p. 196° C. (decomp.): ¹H-NMR (300 MHz, CD₃CN, ppm) 10.8 (1H, br.s), 8.84 (2H, s): ¹³C-NMR (75 MHz, D₂O, ppm) 167.33, 151.19 (t, J=22.4 Hz), 145.44, 133.35, 128.45 (t, J=4.7 Hz): ¹⁹F-NMR (282 MHz, CD₃CN, ppm): 77.65 (1F, quintet, J=151 Hz), 62.30 (4F, d, J=151 Hz): IR (KBr, cm⁻¹) 3113, 2893, 2653, 2526, 1749, 1668, 1629, 1565, 1468, 1417, 1345, 1279, 868, 805, 731. Elemental analysis: Calcd for C₇H₃F₅N₂O₆S: C, 24.86%; H, 0.89%; N, 8.28%. Found: C, 25.13%; H, 0.90%; N, 7.91%.

### Example 5

### Preparation of 2,6-dinitro-4-(pentafluorosulfanyl)benzoic acid (V) using chromium(VI) oxide as an oxidizing agent

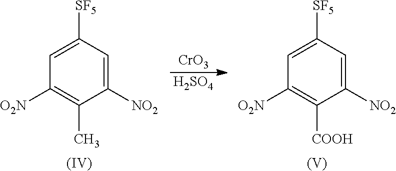

Into a flask equipped with a mechanical stirrer and an bath, were added 200 mL of conc. sulfuric acid and 29.2 g (292 mmol) of chromium(VI) oxide (CrO₃). The mixture was stirred at 20° C. of the bath temperature for 0.5 h. The bath temperature was raised to 30° C. and then 20.0 g of powdered starting material (V) was added into the mixture by five portions in a period of 1.5 h. The mixture was stirred for 5 h at 30° C., cooled to 10° C., and poured into an ice water (1420 g). The resulting solid was collected by filtration, washed with water, and dried in air to give 14.49 g of a white solid. The filtrate was extracted with dichloromethane and then with ethyl acetate. Each of the extracts (dichloromethane and ethyl acetate) was dried over anhydrous magnesium sulfate and filtered. Removal of solvent from each of the filtrates gave 1.96 g and 1.51 g of the respective white solids. The combined white solid (17.96 g) was dissolved in diethyl ether and the ether solution was mixed with a small amount of toluene. After the ether was removed from the mixed solution under reduced pressure, about 20 mL of toluene was added to the solution. The solution was heated moderately, and then the solution was mixed with about 50 mL of hexane and stirred on an ice bath for an hour. The resulting crystals were collected by filtration and washed with hexane, and dried in vacuum to give 12.03 g of the product (V). The filtrate was mixed with hexane to additionally give 1.92 g of the product (V) as white crystals. The total yield of the product (V) was 13.95 g (64%). The physical properties and spectral data of the product are shown in Example 4.

### Example 6

### Preparation and isolation of a stable 1:1 compound of 2,6-dinitro-4-(pentafluorosulfanyl)benzoic acid (V) and isopropanol

2,6-dinitro-4-(pentafluorosulfanyl)benzoic acid (24.06 g) was dissolved in 200 mL of isopropanol by heating. The hot solution was cooled on an ice bath under stifling for an hour. The resulting crystals were collected by filtration, washed with cold isopropanol, and dried in vacuum to give 28.3 g (yield 83%) of a 1:1 compound of 2,6-dinitro-4-(pentafluorosulfanyl)benzoic acid and isopropanol as white needles. The ¹H-NMR analysis and elemental analysis clearly showed that the obtained compound is a 1:1 compound of 2,6-dinitro-4-(pentafluorosulfanyl)benzoic acid and isopropanol. The physical properties and spectral data are shown in the following: M.p.; 197.9-199.1° C. (decomp): ¹H-NMR (300 MHz, CD₃CN, ppm) 8.84 (2H, s), 5.5 (2H, br.s, OH), 3.79-3.91 (1H, m),1.07 (6H, d, J=6.2 Hz): ¹³C-NMR (75 MHz, CD₃CN, ppm) 161.82, 152.77 (m), 147.05, 128.29, 128.06 (m), 63.93, 24.28: ¹⁹F-NMR (282 MHz, CD₃CN, ppm) 77.68 (1F, quintet, J=150.5 Hz), 62.30 (4F, d, J=150.5 Hz): IR (KBr, cm⁻¹) 3444, 3106, 2982, 2525, 2358, 1749, 1585, 1557, 1470, 1347, 1279, 1082, 853, 605. Elemental analysis: Calcd for C₇H₃F₅N₂O₆S—C₃H₇OH: C, 30.16%; H, 2.78%; N, 7.03%. Found: C, 30.10%; H, 2.88%; N, 6.92%.

### Example 7

### Preparation of 1,3-dinitro-5-(pentafluorosulfanyl)benzene (I) by decarboxylation of 2,6-dinitro-4-(pentafluorosulfanyl)benzoic acid (V)

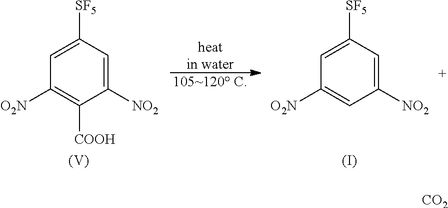

A mixture of 1.0 g (2.96 mmol) of 2,6-dinitro-4-(pentafluorosulfanyl)benzoic acid (V) and 15 mL of water was heated at 16.5 h at 105° C. (bath temperature) and then for 2 h at 120° C. (bath temperature). After cooling, the resulting solid was collected by filtration. The solid was dissolved in dichloromethane. The dichloromethane solution was washed with aqueous saturated sodium bicarbonate solution, dried with magnesium sulfate, and filtered. Removal of solvent gave 829 mg (yield 95%) of 1,3-dinitro-5-(pentafluorosulfanyl)benzene (I) as a light yellow solid. Spectral data of this product agreed with those reported in the literature (U.S. Pat. No. 5,220,070).

### Example 8

### Preparation of 1,3-dinitro-5-(pentafluorosulfanyl)benzene (I) by decarboxylation of 2,6-dinitro-4-(pentafluorosulfanyl)benzoic acid (V) under conditions including a conjugated base of 2,6-dinitro-4-(pentafluorosulfanyl)benzoic acid (V)

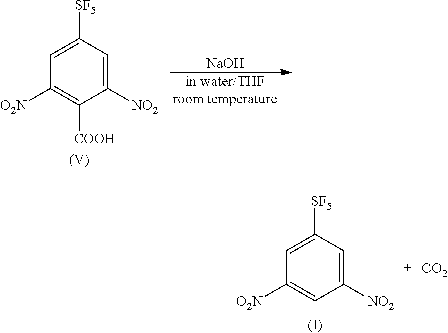

59.1 mL of 0.5M aqueous sodium hydroxide solution was added to a solution of 20.0 g (59.1 mmol) of 2,6-dinitro-4-(pentafluorosulfanyl)benzoic acid (V) in 177 mL of tetrahydrofuran (THF) at room temperature. The mixture was then stirred at room temperature for 18 h. NMR analysis of the reaction mixture showed that the starting material (V) was consumed. The reaction mixture consisted of two layers; the upper layer was a THF layer and the lower layer was a water layer. The water layer was separated and extracted with toluene. The toluene layer was combined with the THF layer. The combined organic layer was washed with aqueous 20% NaCl solution, dried over anhydrous magnesium sulfate, and filtered. Removal of solvent from the filtrate gave 17.1 g of a yellow solid, which was then dissolved in 30 mL of methanol by heating. Water (9.4 mL) was added to the methanol solution under stifling. The mixture was left on standing to room temperature and then cooled on an ice bath with stifling. The resulting crystals were collected by filtration, washed with water, and dried in vacuum to give 16.4 g (yield, 94%) of product (I). Spectral data of this product agreed with those reported by the literature (U.S. Pat. No. 5,220,070).

### Example 9

### Preparation of 1,3-dinitro-5-(pentafluorosulfanyl)benzene (I) from (pentafluorosulfanyl)benzene (VI)

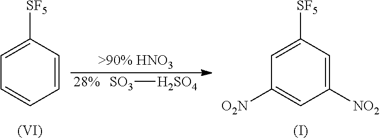

35.0 g (556-500 mmol) of >90% nitric acid was dropwise added to 92.5 g of fuming sulfuric acid (28% SO₃—H₂SO₄) in a 250 mL flask cooled with an ice bath. Into the mixture, 10.2 g (50 mmol) of (pentafluorosulfanyl)benzene (VI) was added dropwise over 1 h. In order to accelerate the reaction, three drops of trifluoroacetic acid were added to the mixture when about a half of amount of (pentafluorosulfanyl)benzene was added. After the addition of (pentafluorosulfanyl)benzene was complete, the reaction mixture was stirred at room temperature for 1 h and heated at 80° C. for 45 h. The reaction mixture was poured into ice-water (400 g) and extracted with dichloromethane. The organic layer was washed with water and then aqueous saturated sodium bicarbonate solution, dried with magnesium sulfate, and filtered. Evaporation of solvent from the filtrate gave a residue, which was column-chromatographed on silica gel using hexane-ethyl acetate (15:1) as an eluent to give 4.98 g (yield 34%) of 1,3-dinitro-5-(pentafluorosulfanyl)benzene (I) as a light yellow solid. Spectral data of this product agreed with those reported by the literature (U.S. Pat. No. 5,220,070).

### Example 10

### Preparation of 1,3-dinitro-5-(pentafluorosulfanyl)benzene (I) from 3-(pentafluorosulfanyl)nitrobenzene (VII)

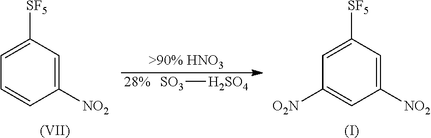

35.0 g (556-500 mmol) of >90% nitric acid was dropwise added to 76.8 g of fuming sulfuric acid (28% SO₃—H₂SO₄) in a 250 mL flask cooled with an ice bath. 3-(Pentafluorosulfanyl)nitrobenzene (VII) (12.5 g, 50 mmol) was added into the mixture, and the reaction mixture was heated at 80° C. for 3 days and then at 88° C. for an additional day. The reaction mixture was poured into ice-water (670 g) and extracted with dichloromethane. The organic layer was washed with water and then aqueous saturated sodium bicarbonate solution, dried with magnesium sulfate, and filtered. Evaporation of solvent from the filtrate gave a residue (8.54 g; crude yield 58%), which was column-chromatographed on silica gel using hexane-ethyl acetate (15:1) as an eluent to give 4.83 g (yield 33%) of 1,3-dinitro-5-(pentafluorosulfanyl)benzene (I) as a light yellow solid. Spectral data of this product agreed with those reported by the literature (U.S. Pat. No. 5,220,070).

### Reference Example 1

### Preparation of 3-(pentafluorosulfanyl)nitrobenzene (VII) from (pentafluorosulfanyl)benzene (VI)

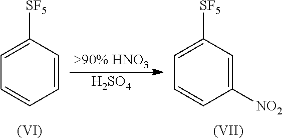

100 g (490 mmol) of (pentafluorosulfanyl)benzene (VI) and 75.6 mL of concentrated sulfuric acid were placed in a 500 mL flask. Into this, was slowly added a mixture of 45.7 mL (1090-980 mmol) of >90% nitric acid and 45.7 mL of concentrated sulfuric acid over 40 min under cooling with a water bath. After the addition, the reaction mixture was stirred at room temperature for 24 h. The reaction mixture was poured into ice-water (300 g) and extracted with dichloromethane. The organic layer was washed with water and then aqueous saturated sodium bicarbonate solution, dried with magnesium sulfate, and filtered. Evaporation of solvent from the filtrate gave 120 g of an oily product, which was distilled under reduced pressure to give 113.9 g (yield 93%) of 3-(pentafluorosulfanyl)nitrobenzene (VII): B.p. 98-100° C./5.4-5.6 mmHg. Spectral data of this product agreed with those reported by the literature [Tetrahedron, Vol. 56 (2000), pp. 3399-3408].

### Reference Example 2

### Preparation of 1,3-diamino-5-(pentafluorosulfanyl)benzene (VIII) from 1,3-dinitro-5-(pentafluorosulfanyl)benzene (I) by reduction with Fe powder/HCl

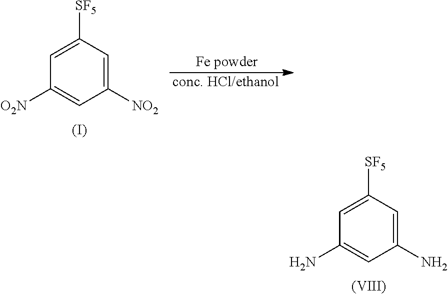

2.3 mL of concentrated hydrochloric acid, 38 mL of ethanol, and 2.0 g (6.8 mmol) of 1,3-dinitro-5-(pentafluorosulfanyl)benzene (I) can be combined in a 100 mL flask. Iron powder (2.28 g, 40.8 mmol) is added into the flask and the reaction mixture heated under reflux for 4 h. After cooling, the mixture is filtered in order to remove remaining iron powder. Removal of ethanol solvent from the filtrate under reduced pressure provides a residue. To the residue, dichloromethane and then 27 mL (27 mmol) of 1M aqueous ammonia solution are added. The resulting gel-like black solid is removed by filtering through celite. The filtrate is extracted with dichloromethane, and the organic layer is dried with magnesium sulfate and filtered. Removal of solvent from the filtrate provides approximately 1.46 g (crude yield 92%) of the product as a solid, which can be recrystallized from diethyl ether-hexane to give 1.18 g (yield 74%) of pure product, 1,3-diamino-5-(pentafluorosulfanyl)benzene (VIII). Spectral data of this product should agree with those reported in the literature (U.S. Pat. No. 5,220,070).

What is claimed is:

1. A process for preparing 1,3-dinitro-5-(pentafluorosulfanyl)benzene, the process comprising: (step 1) reacting 4-(pentafluorosulfanyl)toluene with a nitrating agent to form 2-nitro-4-(pentafluorosulfanyl)toluene; (step 2) reacting the resulting 2-nitro-4-(pentafluorosulfanyl)toluene with a nitrating agent to form 2,6-dinitro-4-(pentafluorosulfanyl)toluene; (step 3) oxidizing the resulting 2,6-dinitro-4-(pentafluorosulfanyl)toluene to form 2,6-dinitro-4-(pentafluorosulfanyl)benzoic acid; and (step 4) decarboxylating the resulting 2,6-dinitro-4-(pentafluorosulfanyl)benzoic acid to form 1,3-dinitro-5-(pentafluorosulfanyl)benzene.

2. The process of claim 1, wherein the step 1 and step 2 are conducted in a one-pot reaction.

3. A process for preparing 1,3-dinitro-5-(pentafluorosulfanyl)benzene, the process comprising decarboxylating 2,6-dinitro-4-(pentafluorosulfanyl)benzoic acid to form 1,3-dinitro-5-(pentafluorosulfanyl)benzene.

4. The process of claim 3, wherein the decarboxylation is conducted under conditions including a conjugated base of 2,6-dinitro-4-(pentafluorosulfanyl)benzoic acid.

5. A process for preparing 2,6-dinitro-4-(pentafluorosulfanyl)benzoic acid, the process comprising: oxidizing 2,6-dinitro-4-(pentafluorosulfanyl)toluene to form 2,6-dinitro-4-(pentafluorosulfanyl)benzoic acid.

6. A process for preparing 2,6-dinitro-4-(pentafluorosulfanyl)toluene, the process comprising: reacting 4-(pentafluorosulfanyl)toluene with a nitrating agent to form 2,6-dinitro-4-(pentafluorosulfanyl)toluene.

7. The process of claim 6, wherein the reaction is conducted at less than 80° C.

8. A process for preparing 2,6-dinitro-4-(pentafluorosulfanyl)toluene, the process comprising: reacting 2-nitro-4-(pentafluorosulfanyl)toluene with a nitrating agent to form 2,6-dinitro-4-(pentafluoro sulfanyl)toluene.

9. The process of claim 8, wherein the reaction is conducted at less than 80° C.

10. A process for preparing 2-nitro-4-(pentafluorosulfanyl)toluene, the process comprising: reacting 4-(pentafluorosulfanyl)toluene with a nitrating agent to form 2-nitro-4-(pentafluorosulfanyl)toluene.

11. The process of claim 10, wherein the reaction is conducted at less than 80° C.

12. A (pentafluorosulfanyl)benzene derivative of formula (A): 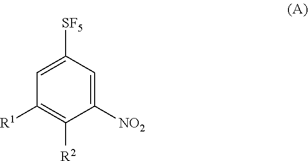
in which R¹ is a hydrogen atom or a nitro group and R² is a methyl group or a carboxyl group.

13. The derivative of claim 12, wherein the (pentafluorosulfanyl)benzene derivative is selected from a group consisting of 2,6-dinitro-4-(pentafluorosulfanyl)benzoic acid, 2,6-dinitro-4-(pentafluorosulfanyl)toluene, and 2-nitro-4-(pentafluorosulfanyl)toluene.

14. A 1:1 compound of 2,6-dinitro-4-(pentafluorosulfanyl)benzoic acid and isopropanol.

15. A process for preparing 1,3-dinitro-5-(pentafluorosulfanyl)benzene, the process comprising: reacting 3-(pentafluorosulfanyl)nitrobenzene with a nitrating agent to form 1,3-dinitro-5-(pentafluoro sulfanyl)benzene.

16. The process of claim 15, wherein the reaction is conducted at less than 100° C.

17. A process for preparing 1,3-dinitro-5-(pentafluorosulfanyl)benzene, the process comprising: reacting (pentafluorosulfanyl)benzene with a nitrating agent to form 1,3-dinitro-5-(pentafluorosulfanyl)benzene.

18. The process of claim 17, wherein the reaction is conducted at less than 100° C.
